(19) United States
(12) Patent Application Publication
(10) Pub. No.: US 2016/0101294 A1
(43) Pub. Date: Apr. 14, 2016

(54) PORTABLE PHOTOTHERAPY APPARATUS FOR PSORIASIS

(71) Applicant: NATIONAL CENTRAL UNIVERSITY, TAOYUAN COUNTY (TW)

(72) Inventors: CHING-CHERNG SUN, TAOYUAN COUNTY (TW); SZU-YU CHEN, TAOYUAN COUNTY (TW); YU-YU CHANG, KAOHSIUNG CITY (TW); RODOLFO GARCIA CAMACHO, TAOYUAN COUNTY (TW)

(21) Appl. No.: 14/513,544

(22) Filed: Oct. 14, 2014

(51) Int. Cl. A61N 5/06

(52) U.S. Cl. CPC A61N 5/0616; A61N 2005/0626; A61N 2005/0651; A61N 2005/0661; A61N 2005/0663

(57) ABSTRACT

A portable phototherapy apparatus for treating a skin disorder in a patient. The apparatus includes an illumination source, an image capture device, a projecting system and a controller. The illumination source is configured to irradiate the patient's skin. The image capture device for capturing an image of lesion areas of the patient's skin needing treatment. The projecting system includes a therapeutic light source for generating UV radiation. The controller configured to cause the projecting system to form a shaped treatment image comprising the UV radiation on the patient's skin based on the image of the lesion areas captured by the image capture device such that the therapeutic light source delivers a dose of the UV radiation only to the lesion areas of the skin.

### BACKGROUND OF THE INVENTION

1. Field of the Invention

The present invention relates to an ultraviolet phototherapy apparatus for treatment of skin disorders, such as psoriasis or the like, and is particularly concerned with a portable UV phototherapy apparatus for distributing ultraviolet irradiation dosage in a treatment pattern such that only plaques of psoriasis receive a therapeutic exposure dose of UV radiation.

2. Description of the Related Art

Psoriasis is a non-contagious chronic skin disease, which is typically characterized by thick red zones of the skin which are covered with silvery or whitish scales. Th e extent of the disease is variable, with small local areas or the whole of the surface of the body being affected.

Phototherapy has a long history of treating psoriasis dating back to 1926 when natural solar treatments such as the Goekerman regimen and Heliotherapy (sunlight rich in UVB at the Dead Sea) were practiced. However, these natural solar treatments have mostly given way to modern booths or chambers that provide artificial UVA and/or UVB radiation.

Specifically, it is known to treat psoriasis by exposing the affected area of the skin to a narrow range (approximately 300-330 nm) of ultraviolet radiation, namely, to radiation in the upper portion of the so-called UVB range (280 to 315 nm) and in the lower portion of the so-called UVA range (315 to 400 nm). The spectral zone between approximately 300 and 330 nm includes radiation which contains sufficient amounts of quantum energy for the skin treatment and is capable of penetrating into the epidermis to reach the inner boundary of the epidermis, i.e., the stratum which is the staging point of psoriasis. Although the benefits of UVA and UVB are known in psoriasis treatment, the adverse affects upon healthy tissue, particularly of UVB radiation, are also well known and a medical concern.

Most of the devices designed for phototherapy, both in the UVA and UVB, are table top projectors for irradiating the face or feet, or booth or chamber types of devices. The devices rely upon tubular fluorescent or tubular mercury bulbs as UV sources. The booths are generally composed of multiple banks of bulbs, and irradiate large areas, usually the whole body. Thus, in traditional phototherapy, substantially all of a patient's skin is exposed to the radiation. Although large unaffected portions of the body can be protected with draping or wrapping materials, but this is impractical for most clinical use, not to mention for personal use.

It has been demonstrated that some lesional tissue (psoriatic plaque for example) can withstand much more UVB radiation than healthy tissue. This is largely due to the thickness of the plaque areas. However, the radiation delivered to the plaque in booth therapy is limited to the amount of radiation that the adjacent health tissue can withstand. In addition, since the radiation may be provided to both healthy and lesional tissue, it could lead to unnecessary UV dosage accumulated in the normal skin. Another shortcoming is that since the intensity within the exposed area of the skin is not uniform, the treatment efficacy can be diverse.

### SUMMARY OF THE INVENTION

Accordingly, it is an object of the present invention to provide a phototherapy apparatus that can optimize the phototherapy in question as the therapy targets only the affected skin, increasing the effectiveness of the therapy.

It is another object of the present invention to provide a portable phototherapy apparatus that is automated and small in size for personal use.

Thus, the present invention is directed to a phototherapy apparatus for treating a skin disorder in a patient, which comprises generally an illumination source, an image capture device, a projecting system and a controller. The illumination source is configured to irradiate the patient's skin. The image capture device is provided for capturing an image of lesion areas of the patient's skin needing treatment. The projecting system includes a therapeutic light source with at least one UV light emitting diode for generating UV radiation. The controller i s configured to cause the projecting system to form a shaped treatment image comprising the UV radiation on the patient's skin based on the image of the lesion areas captured by the image capture device such that the therapeutic light source delivers a dose of the UV radiation only to the lesion areas of the skin.

Preferably, the projecting system further includes a beam-shaping device, such as a spatial light modulator, provided to receive the UV radiation from the therapeutic light source and transform the UV radiation to the shaped treatment image.

These and other features and advantages of the present invention will become apparent to those skilled in the art upon a reading of the following detailed description when taken in conjunction with the drawings wherein there is shown and described an illustrative embodiment of the invention.

### BRIEF DESCRIPTION OF THE DRAWINGS

FIG. 1 is a cross section of a phototherapy apparatus in accordance with the preferred embodiment of the present invention;

FIG. 2 is a top view of the phototherapy apparatus; FIG. 3 is a block diagram of the phototherapy apparatus; and

FIG. 4 is a perspective view of the phototherapy apparatus and the body under treatment.

### DETAILED DESCRIPTION OF PREFERRED EMBODIMENT

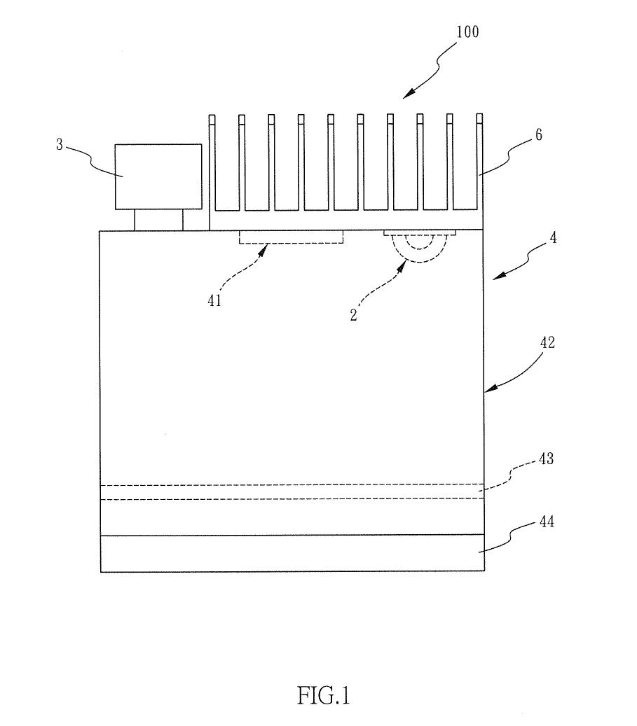

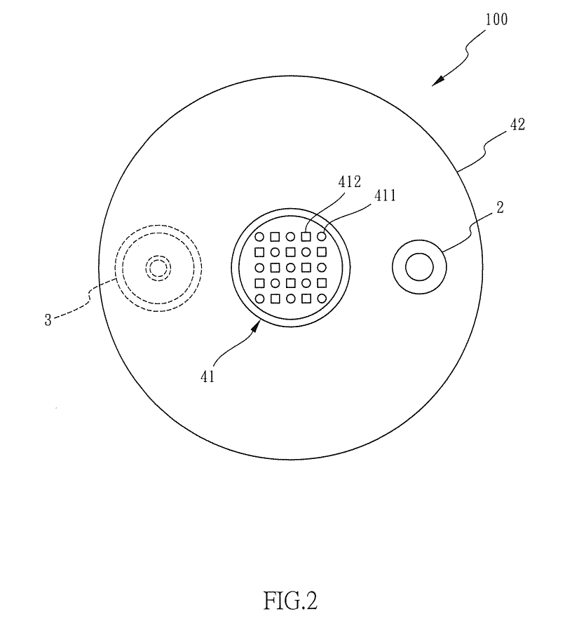

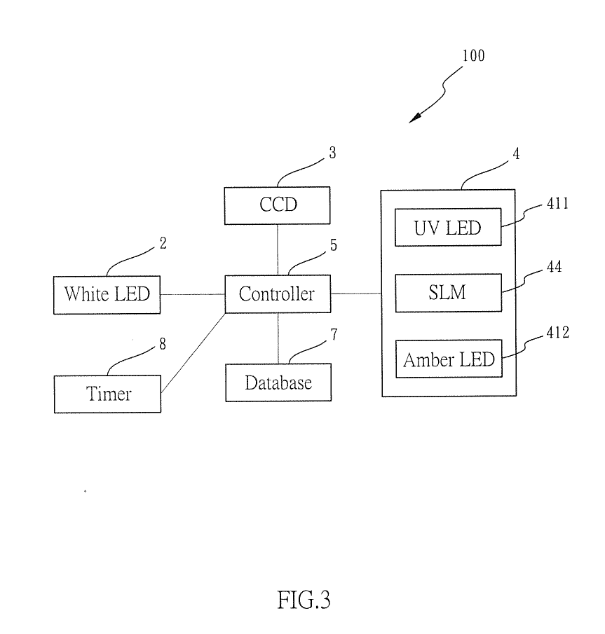

Referring now to the drawings, and particularly to FIGS. 1 and 2, there is shown a portable phototherapy apparatus 100 for treating a skin disorder, such as psoriasis or the like, according to the invention, the apparatus 100 generally comprising a base 1, an illumination source 2 disposed on the base 1, an image capture device 3 joined to the base 1, a projecting system 4 mounted on the base 1 and a controller 5 (FIG. 3) wirely or wirelessly connected to the illumination source 2, the image capture device 3 and the projecting system 4.

The illumination source 2 may be a white light emitting diode (white LED) for irradiating the patient's skin. Preferably, the illumination source 2 has a color rendering index of higher than 95. With the high CRI of the illumination radiation, the image capture device 3, such as a charge-coupled device (CCD), can capture an image L of lesion areas of the patient's skin with true color. Thus, a shade of red of the lesion areas can be well identified from the spectral information of the image L of the lesion areas.

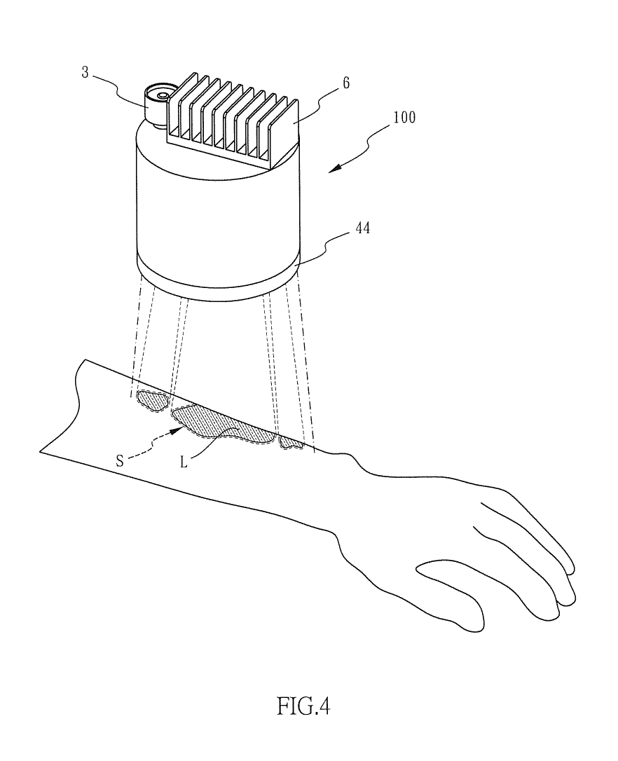

The projecting system 4 includes a therapeutic light source 41 that has at least one UV light emitting diode 411 for generating UV radiation. The therapeutic light source 41 along with the illumination source 2 is mounted on a heat sink 6 for heat dissipation. With reference to FIG. 3, the controller 5 is configured to cause the projecting system 4 to form a shaped treatment image S (see FIG. 4) comprising the UV radiation on the patient's skin based on the image L of the lesion areas captured by the image capture device 3, such that the therapeutic light source 4 delivers a dose of the UV radiation only to the lesion areas of the skin.

Referring back to FIG. 1, more specifically, the projecting system 4 further includes a collimating optics 42, a longpass filter 43 and a beam-shaping device 44. The collimating optics 42 is interposed in between the therapeutic light source 41 and the beam-shaping device 43 for guiding the light generated from the therapeutic light source 41 toward the beam-shaping device 43. Th e longpass filter 43 is provided to remove substantially all light below 300 nm that is harmful to skin. The beam-shaping device 44, such as a spatial light modular or a liquid-crystal on silicon (LcoS), is provided to receive the UV radiation from the therapeutic light source 41 and transform the UV radiation to the shaped treatment image S.

In this embodiment, the therapeutic light source 41 includes an array of UV light emitting diodes 411 for generating the UV radiation and an array of amber light emitting diodes 412 for generating the amber radiation. It is understood that the UV light emitting diodes 411 can treat the skin diseases including psoriasis. Preferably, the UV radiation has a wavelength band in a range of about 300-350 nm, and more preferably, the UV radiation has a peak wavelength of about 310 nm. On the other hand, the amber radiation helps the affected skin to have a shorter recovery period. The amber light emitting diodes 412 may be controlled by the controller 5 to irradiate the patient's skin after the UV radiation treatment is finished. Preferably, the wavelength of the amber radiation is in a range of about 570-610 nm.

Referring to FIG. 3, the controller 5 is also coupled to the image capture device 3 to analyze and identify the shade of red in the image L of the lesion areas and thereby determine a severity degree of the lesion areas based at least on the shade of red in the image L of the lesion areas. A database 7 may be employed to store a plurality of medical information where the shades of red, the severity degrees, and the doses are all correlated. With the medical information from the database 7, the controller 5 can cause the therapeutic light source 41 delivers an appropriate dose of the UV radiation to the affected skin according to the severity degree of the lesion areas. Moreover, a timer 8, which is coupled to the controller 5, may be employed to control a dosage time so that the timer 8 turns off the therapeutic light source 41 once the treatment time ends.

What is claimed is:

1. A phototherapy apparatus for treating a skin disorder in a patient, the apparatus comprising:
an illumination source configured to irradiate the patient's skin;
an image capture device for capturing an image of lesion areas of the patient's skin needing treatment;
a projecting system comprising a therapeutic light source that includes at least one UV light emitting diode for generating UV radiation; and
a controller configured to cause the projecting system to form a shaped treatment image comprising the UV radiation on the patient's skin based on the image of the lesion areas captured by the image capture device such that the therapeutic light source delivers a dose of the UV radiation only to the lesion areas of the skin.

2. A phototherapy apparatus as recited in claim 1, wherein the projecting system further includes a beam-shaping device configured to receive the UV radiation from the therapeutic light source and transform the UV radiation to the shaped treatment image.

3. A phototherapy apparatus as recited in claim 2, wherein the projecting system further includes a collimating optics interposed in between the therapeutic light source and the beam-shaping device.

4. A phototherapy apparatus as recited in claim 2, wherein the beam-shaping device is a spatial light modular.

5. A phototherapy apparatus as recited in claim 2, wherein the beam shaping device is a liquid-crystal on silicon (LcoS) device.

6. A phototherapy apparatus as recited in claim 1, wherein the at least one UV light emitting diode of the therapeutic light source includes an array of UV light emitting diodes for generating the UV radiation.

7. A phototherapy apparatus as recited in claim 1, therapeutic light source further includes at least one amber light emitting diode for generating an amber radiation with a wavelength range of about 570-610 nm, wherein the controller is further configured to cause the amber light emitting diode to irradiate the patient's skin for recovery after the UV radiation treatment is finished.

8. A phototherapy apparatus as recited in claim 1, wherein the skin disorder is psoriasis.

9. A phototherapy apparatus as recited in claim 8, wherein the projecting system further comprising a longpass filter which removes substantially all light below 300 nm.

10. A phototherapy apparatus as recited in claim 8, wherein the UV radiation has a wavelength band in a range of about 300-350 nm.

11. A phototherapy apparatus as recited in claim 10, wherein the UV radiation has a peak wavelength of about 310 nm.

12. A phototherapy apparatus as recited in claim 8, wherein the controller is also configured to identify a shade of red in the image of the lesion areas and thereby determine a severity degree of the lesion areas based at least on the shade of red in the image of the lesion areas, and further to cause the therapeutic light source delivers the dose of the UV radiation according to the severity degree of the lesion areas.

13. A phototherapy apparatus as recited in claim 12, wherein the illumination source is a white LED light source with a color rendering index of higher than 95.

14. A phototherapy apparatus as recited in claim 12, further comprising a timer for control a dosage time.
